(19) United States
(12) Patent Application Publication
(10) Pub. No.: US 2017/0342479 A1
(43) Pub. Date: Nov. 30, 2017

(54) METHODS OF HUMAN LEUKOCYTE ANTIGEN TYPING

(71) Applicant: HUMAN LONGEVITY, INC., San Diego, CA (US)

(72) Inventor: Chao XIE, Singapore (SG)

(21) Appl. No.: 15/605,767

(22) Filed: May 25, 2017

Related U.S. Application Data
(60) Provisional application No. 62/342,817, filed on May 27, 2016.

(51) Int. Cl. C12Q 1/68; G06F 19/22

(52) U.S. Cl. CPC C12Q 1/6869; G06F 19/22; C12Q 1/6881; C12Q 2600/156

(57) ABSTRACT

Described herein are methods, systems, and media for HLA typing an individual from nucleic acid or protein sequences. The methodology disclosed herein represents significant improvements over current methods of HLA typing.

### CROSS-REFERENCE TO RELATED APPLICATIONS

This application claims priority to U.S. Application Ser. No. 62/342,817, filed on May 27, 2016, which is incorporated by reference herein in its entirety.

### BACKGROUND OF THE INVENTION

Every year an estimated 30,000 patients in the U.S. receive organ transplants and another 20,000 receive bone-marrow transplants. Organ disease is a leading cause of mortality and every 10 minutes another individual is added to an organ donation waiting list. A main cause of failure after organ transplantation is rejection by an individual's immune system. In order to minimize the chances of rejection doctors routinely test donors and recipients for a match at their respective human leukocyte antigen (HLA) alleles, sometimes known as the major histocompatibility complex (MEW). The proteins encoded by the HLA locus are cell surface transmembrane proteins that present peptides derived from both self and foreign antigens. Different HLA molecules differ in their presentation of self and foreign antigens. It is these self-peptides that instruct the recipient's T-cell and B-cell repertoire and set the repertoire to tolerate the recipient's own organs. When encountered with an organ from a donor that is not an HLA “match” the recipient's own T cells and B cells will attack the organ as though it were a foreign pathogen leading to rejection and poor outcomes. There is currently a long-standing need for improved methods of patient HLA typing.

### SUMMARY OF THE INVENTION

The methods, systems and media of this disclosure represent a substantial improvement on current HLA typing methods based on current next-generation DNA sequencing technologies. The problem with the use of current DNA sequencing technologies for HLA typing is that these technologies create short sequence reads that greatly increase the difficulty in determining the sequence of highly polymorphic loci—such as the HLA locus. The improvements, detailed herein, are reflected by increased accuracy, efficiency, and speed compared to existing methods. As shown in FIG. 1, compared to current methods, the methods described herein result in more accurate results for both Class I and Class II HLA typing when compared to other methods. Current methods of HLA typing from an individual's DNA are of limited usefulness due to inefficiency of the methods implemented to arrive at the results and a lack accuracy of said results once obtained. Referring to FIG. 1 methods known as HLA-VBSeq, SOAP HLA and HLA*PRG require enormous computer processing resources and significant wait times for results. Optitype, while operating more efficiently, sacrifices accuracy and usability. See e.g., Szolek et al. OptiType: precision HLA typing from next-generation sequencing data. Bioinformatic, Vol. 30 no. 23 2014, pages 3310-3316. Optitype pre-filters its database of HLA reference sequences compromising accuracy thus, for example, wrongly types people with rare HLA alleles, and does not even attempt to align class II HLA reference sequences, limiting its usefulness in a clinical setting. In order for DNA-based HLA typing to contribute to advancements in clinical treatment new methods, such as those described herein, must be developed. In certain embodiments, the methods described herein do not prefilter HLA reference sequences, or reduce the amount of HLA alleles tested using the algorithm.

Still referring to FIG. 1, in certain instances, the methods, systems, and media, described herein result in reduced runtime compared to current HLA typing methods. In certain embodiments, the application runtime is reduced 2, 3, 4, 5, 6, 7, 8, 9, or 10 times or more compared to any of the HLA-VBSeq, SOAP HLA and HLA*PRG, or Optitype method. In certain instances, the methods, systems, and media, described herein result in reduced processor utilization compared to current HLA typing methods. In certain embodiments, the application results in processor utilization that is reduced 2, 3, 4, 5, 6, 7, 8, 9, or 10 times or more compared to any of the HLA-VBSeq, SOAP HLA and HLA*PRG, or Optitype method.

Referring to FIG. 2, the HLA locus resides on human chromosome 6. Since chromosome 6 is an autosome, each individual possesses two copies of each HLA gene. Genes of the HLA class I locus that are important for tissue compatibility are denoted A, B and C; genes of the class II locus that are important for tissue compatibility are denoted DR, DQ, and DP. Referring to FIG. 3, each one of these genes has hundreds or thousands of total genetic alleles. The genetic alleles can be further grouped into their 4-digit type. The difference between an allele and 4-digit allele is that the 4-digit allele captures the differences in the DNA sequence that lead to differences in protein sequence. For example, alleles may be distinguishable based upon a variant in their DNA sequence, but if this variant is a synonymous variant, then it will not lead to an amino acid change in the expressed protein, making them functionally and immunologically indistinguishable. Still referring to FIG. 3, each HLA gene has multiple exons, of these, exons 2 and 3 for class I genes, and exon 2 for class II genes are core exons. The core exons are so named because they contain the peptide binging pocket or peptide binding core for the HLA molecule. All HLA genes in the IMGT reference database have had their core exons sequenced.

FIG. 4 illustrates the standard convention for HLA naming. Each gene has a letter designation followed by an asterisk. Field one denotes the allele group while field two denotes the specific HLA protein. Together, field one and field two represent the 4-digit HLA allele. The remaining fields field 3 and field 4 are used to show differences in the DNA sequence that do not translate to changes in amino acid sequence of the resulting protein. Field three represents synonymous variants located in the coding region, while field four represents variants in non-coding regions.

FIG. 5 illustrates the main difficulty in sequencing the HLA locus. Primarily, due to the high degree of homology present in the locus, one sequence read can map to many different genes, especially when deploying a short read technology. Even worse, reads from HLA genes can map to wrong genes on the human reference genome, because the reference genome can only represent one type of any HLA gene.

FIG. 6 shows an alignment matrix and highlights this problem when a plurality of DNA sequence reads are present. Each of the plurality of reads can map to multiple HLA alleles further confounding and slowing down the task of unambiguously mapping a plurality of DNA sequence reads to alleles.

In a certain embodiment, the disclosure provides a method of determining an individual's 4-digit human leukocyte antigen (HLA) allele composition, the method comprising: mapping at least one nucleic acid sequence read from the individual to known HLA allele reference sequences to identify a first set of HLA alleles that explain the at least one nucleic acid sequence read; and using a multiple sequence alignment (MSA) of known HLA allele reference sequences to identify one or more additional HLA allele reference sequences that match the at least one nucleic acid sequence read equally well as the first set of HLA alleles; wherein the individual's 4-digit HLA allele composition comprises the one or more additional HLA allele reference sequences that have the closest match to the at least one nucleic acid sequence read from the individual. In certain embodiments, the nucleic acid is DNA. The at least one nucleic acid sequence read can be obtained by a next-generation sequencing technique. The at least one nucleic acid sequence read can be less than 300 nucleotides. The at least one nucleic acid sequence read can be a plurality of nucleic acid sequence reads. The multiple sequence alignment can comprise all known HLA allele reference sequences. The multiple sequence alignment can comprise all known HLA allele reference sequences available from IMGT/HLA database. The first set of HLA alleles can comprise HLA alleles with at least 95% identity to the at least one nucleic acid sequence read from the individual. The one or more additional HLA allele reference sequences can comprise HLA alleles with at least 95% identity to the at least one nucleic acid sequence read from the individual. In some instances, the method further comprises generating a solution set and a comparison set, wherein the solution set comprises the one or more additional HLA allele reference sequences that have the closest match to the at least one nucleic acid sequence read from the individual based upon core exons and the comparison set comprises HLA allele reference sequences that performed nearly as well those of the solution set. The core exons can consist of exons 2 and 3 if the HLA allele reference sequence is a class I molecule. The core exons can consist of exon 2 if the HLA allele reference sequence is a class II molecule. In some instances, the method further comprises comparing each of the HLA allele reference sequences of the solution set with one or more HLA allele reference sequences of the comparison set based upon all shared exons, wherein the solution set is updated with an HLA allele reference sequence from the comparison set if one or more of the HLA allele reference sequences from the comparison set better explains the nucleic acid sequence reads from the individual. The method can be repeated more than once. The method can be repeated until no HLA allele reference sequence from the solution set can be replaced by an HLA allele from the comparison set. In certain instances, only nucleic acid sequence reads mapped to an HLA reference sequence of the solution set or to an HLA reference sequence of the comparison set, but not to both, and not to any other HLA allele reference sequence in the solution set are used to evaluate whether the putative HLA allele should be replaced by the comparison allele. The method can be repeated more than once. The method can be repeated until no HLA allele reference sequence from the solution set can be replaced by an HLA allele from the comparison set. In certain instances, the method further comprises checking zygosity, wherein checking zygosity determines whether an individual is heterozygous or homozygous for any one or more HLA alleles of the individual's 4-digit HLA allele composition. Checking zygosity may comprise counting the at least one nucleic acid sequence reads that map to each allele of a given HLA gene. In certain instances, the individual is determined to be homozygous if the amount of sequence reads is at least 2 times or more than the next most strongly correlated allele. In certain instances, the method further comprises determining a full resolution HLA composition, wherein determining the full resolution HLA composition comprises extracting the at least one nucleic acid sequence read that unambiguously align to an individual's 4-digit HLA allele composition and aligning the at least one nucleic acid sequence read all HLA allele reference sequences that are contained within the 4-digit HLA allele group. In certain instances, the individual's 4-digit HLA allele composition is the major histocompatibility complex (MHC) class I allele composition. In certain instances, the individual's 4-digit HLA allele composition is the major histocompatibility complex (MHC) class II allele composition. In certain instances, the individual's 4-digit HLA allele composition is the major histocompatibility class I and the major histocompatibility class II allele composition. In certain instances, the method is performed using a computer and runtime is reduced by at least three-fold compared with a computer running the Optitype method. The method is useful for an individual suffering from an autoimmune disease. The method is useful for an individual in need of an organ transplant.

In another embodiment, the disclosure provides for a method of determining an individual's 4-digit human leukocyte antigen (HLA) allele composition, the method comprising: mapping at least one amino acid sequence translated from at least one nucleic acid sequence read from the individual to known HLA allele reference sequences to identify a first set of HLA alleles that explain the at least one amino acid sequence; and using a multiple sequence alignment (MSA) of known HLA allele reference sequences to identify one or more additional HLA allele reference sequences that match the at least one amino acid sequence equally well as the first set of HLA alleles; wherein the individual's 4-digit HLA allele composition comprises the one or more additional HLA allele reference sequences that have the closest match to the at least one amino acid translated from at least one nucleic acid sequence read from the individual. In certain embodiments, the nucleic acid is DNA. The at least one nucleic acid sequence read can be obtained by a next-generation sequencing technique. The at least one nucleic acid sequence read can be less than 300 nucleotides. The at least one nucleic acid sequence read can be a plurality of nucleic acid sequence reads. The multiple sequence alignment can comprise all known HLA allele reference sequences. The multiple sequence alignment can comprise all known HLA allele reference sequences available from IMGT/HLA database. The first set of HLA alleles can comprise HLA alleles with at least 95% identity to the at least one nucleic acid sequence read from the individual. The one or more additional HLA allele reference sequences can comprise HLA alleles with at least 95% identity to the at least one nucleic acid sequence read from the individual. In some instances, the method further comprises generating a solution set and a comparison set, wherein the solution set comprises the one or more additional HLA allele reference sequences that have the closest match to the at least at least one amino acid sequence based upon core exons and the comparison set comprises HLA allele reference sequences that performed nearly as well those of the solution set. The core exons can consist of exons 2 and 3 if the HLA allele reference sequence is a class I molecule. The core exons can consist of exon 2 if the HLA allele reference sequence is a class II molecule. In some instances, the method further comprises comparing each of the HLA allele reference sequences of the solution set with one or more HLA allele reference sequences of the comparison set based upon all shared exons, wherein the solution set is updated with an HLA allele reference sequence from the comparison set if one or more of the HLA allele reference sequences from the comparison set better explain the sequence data from the individual. The method can be repeated more than once. The method can be repeated until no HLA allele reference sequence from the solution set can be replaced by an HLA allele from the comparison set. In certain instances, only amino acid sequences mapped to an HLA reference sequence of the solution set or to an HLA reference sequence of the comparison set, but not to both, and not to any other HLA allele reference sequence in the solution set are used to evaluate whether the putative HLA allele should be replaced by the comparison allele. The method can be repeated more than once. The method can be repeated until no HLA allele reference sequence from the solution set can be replaced by an HLA allele from the comparison set. In certain instances, the method further comprises checking zygosity, wherein checking zygosity determines whether an individual is heterozygous or homozygous for any one or more HLA alleles of the individual's 4-digit HLA allele composition. Checking zygosity can comprise counting the amino acid sequences that map to each allele of a given HLA gene. In certain instances, the individual is determined to be homozygous if the amount of amino acid sequence is at least 2 times or more than the next most strongly correlated allele. In certain instances, the method further comprises determining a full resolution HLA composition, wherein determining the full resolution HLA composition comprises extracting the at least one amino acid sequence read that unambiguously align to an individual's 4-digit HLA allele composition and aligning the at least one amino acid sequence read all HLA allele reference sequences that are contained within the 4-digit HLA allele group. In certain instances, the individual's 4-digit HLA allele composition is the major histocompatibility complex (MHC) class I allele composition. In certain instances, the individual's 4-digit HLA allele composition is the major histocompatibility complex (MHC) class II allele composition. In certain instances, the individual's 4-digit HLA allele composition is the major histocompatibility class I and the major histocompatibility class II allele composition. In certain instances, the method is performed using a computer and runtime is reduced by at least three-fold compared with a computer running the Optitype method. The method is useful for an individual suffering from an autoimmune disease. The method is useful for an individual in need of an organ transplant.

In another embodiment, described herein, is a non-transitory computer-readable storage media encoded with a computer program including instructions executable by a processor to create an application for determining an individual's 4-digit human leukocyte antigen (HLA) allele composition, the application comprising: a software module mapping at least one nucleic acid sequence read from the individual to known HLA allele reference sequences to identify a first set of HLA alleles that explain the at least one nucleic acid sequence reads; and a software module using a multiple sequence alignment (MSA) of known HLA allele reference sequences to identify one or more additional HLA allele reference sequences that match the at least one nucleic acid sequence read equally well as the first set of HLA alleles; wherein the individual's 4-digit HLA allele composition comprises the one or more additional HLA allele reference sequences that have the closest match to the at least one nucleic acid sequence read from the individual. In certain embodiments, the nucleic acid is DNA. The at least one nucleic acid sequence read can be obtained by a next-generation sequencing technique. The at least one nucleic acid sequence read can be less than 300 nucleotides. The at least one nucleic acid sequence read can be a plurality of nucleic acid sequence reads. The multiple sequence alignment can comprise all known HLA allele reference sequences. The multiple sequence alignment can comprise all known HLA allele reference sequences available from IMGT/HLA database. The first set of HLA alleles can comprise HLA alleles with at least 95% identity to the at least one nucleic acid sequence read from the individual. The one or more additional HLA allele reference sequences can comprise HLA alleles with at least 95% identity to the at least one nucleic acid sequence read from the individual. The application can further comprise a software module generating a solution set and a comparison set, wherein the solution set comprises the one or more additional HLA allele reference sequences that have the closest match to the at least one nucleic acid sequence read from the individual based upon core exons and the comparison set comprises HLA allele reference sequences that performed nearly as well those of the solution set. The core exons can consist of exons 2 and 3 if the HLA allele reference sequence is a class I molecule. The core exons can consist of exon 2 if the HLA allele reference sequence is a class II molecule. The application can further comprise a software module comparing each of the HLA allele reference sequences of the solution set with one or more HLA allele reference sequences of the comparison set based upon all shared exons, wherein the solution set is updated with an HLA allele reference sequence from the comparison set if one or more of the HLA allele reference sequences from the comparison set better explains the at least one at least one nucleic acid sequence read from the individual. The application can be repeated more than once. The application can be run repetitively until no HLA allele reference sequence from the solution set can be replaced by an HLA allele from the comparison set. In some instances, only nucleic acid sequence reads mapped to an HLA reference sequence of the solution set or to an HLA reference sequence of the comparison set, but not to both, and not to any other HLA allele reference sequence in the solution set are used to evaluate whether the putative HLA allele should be replaced by the comparison allele. The application can be repeated more than once. The application can be run repetitively until no HLA allele reference sequence from the solution set can be replaced by an HLA allele from the comparison set. The application can further comprise a software module checking zygosity, wherein checking zygosity determines whether an individual is heterozygous or homozygous for any one or more HLA alleles of the individual's 4-digit HLA allele composition. Checking zygosity can comprise counting the at least one nucleic acid sequence read that maps to each allele of a given HLA gene. In certain instances, the individual is determined to be homozygous if the amount of sequence reads at least 2 times or is 5 times more than the next most strongly correlated allele. The application can further comprise a software module determining a full resolution HLA composition, wherein determining the full resolution HLA composition comprises extracting the at least one nucleic acid sequence read that unambiguously align to an individual's 4-digit HLA allele composition and aligning the at least one nucleic acid sequence read all HLA allele reference sequences that are contained within the 4-digit HLA allele group. In certain instances, the individual's 4-digit HLA allele composition is the major histocompatibility complex (MHC) class I allele composition. In certain instances, the individual's 4-digit HLA allele composition is the major histocompatibility complex (MHC) class II allele composition. In certain instances, the individual's 4-digit HLA allele composition is the major histocompatibility class I and the major histocompatibility class II allele composition. In certain instances, the application results in at least a three-fold reduced runtime by at least three-fold compared with an application running the Optitype method. The application is useful for an individual suffering from an autoimmune disease. The application is useful for an individual in need of an organ transplant.

In another embodiment, described herein, is a non-transitory computer-readable storage media encoded with a computer program including instructions executable by a processor to create an application for determining an individual's 4-digit human leukocyte antigen (HLA) allele composition, the application comprising: a software module mapping at least one amino acid sequence translated from at least one nucleic acid sequence read from the individual to known HLA allele reference sequences to identify a first set of HLA alleles that explain the at least one amino acid sequence; and a software module using a multiple sequence alignment (MSA) of known HLA allele reference sequences to identify one or more additional HLA allele reference sequences that match the at least one amino acid sequence equally well as the first set of HLA alleles; wherein the individual's 4-digit HLA allele composition comprises the one or more additional HLA allele reference sequences that have the closest match to the at least one amino acid translated from at least one nucleic acid sequence read from the individual. In certain embodiments, the nucleic acid is DNA. The at least one nucleic acid sequence read can be obtained by a next-generation sequencing technique. The at least one nucleic acid sequence read can be less than 300 nucleotides. The at least one nucleic acid sequence read can be a plurality of nucleic acid sequence reads. The multiple sequence alignment can comprise all known HLA allele reference sequences. The multiple sequence alignment can comprise all known HLA allele reference sequences available from IMGT/HLA database. The first set of HLA alleles can comprise HLA alleles with at least 95% identity to the at least one nucleic acid sequence read from the individual. The one or more additional HLA allele reference sequences can comprise HLA alleles with at least 95% identity to the at least one nucleic acid sequence read from the individual. The application can further comprise a software module generating a solution set and a comparison set, wherein the solution set comprises the one or more additional HLA allele reference sequences that have the closest match to the at least at least one amino acid sequence based upon core exons and the comparison set comprises HLA allele reference sequences that performed nearly as well those of the solution set. The core exons can consist of exons 2 and 3 if the HLA allele reference sequence is a class I molecule. The core exons can consist of exon 2 if the HLA allele reference sequence is a class II molecule. The application can further comprises a software module for comparing each of the HLA allele reference sequences of the solution set with one or more HLA allele reference sequences of the comparison set based upon all shared exons, wherein the solution set is updated with an HLA allele reference sequence from the comparison set if one or more of the HLA allele reference sequences from the comparison set better explains the at least one amino acid sequences. The application can be repeated more than once. The application can be run repetitively until no HLA allele reference sequence from the solution set can be replaced by an HLA allele from the comparison set. In some instances, only nucleic acid sequence reads mapped to an HLA reference sequence of the solution set or to an HLA reference sequence of the comparison set, but not to both, and not to any other HLA allele reference sequence in the solution set are used to evaluate whether the putative HLA allele should be replaced by the comparison allele. The application can be repeated more than once. The application can be run repetitively until no HLA allele reference sequence from the solution set can be replaced by an HLA allele from the comparison set. The media of any of claims 79 to 95, wherein the application further comprises a software module checking zygosity, wherein checking zygosity determines whether an individual is heterozygous or homozygous for any one or more HLA alleles of the individual's 4-digit HLA allele composition. Checking zygosity can comprise counting the amino acid sequences that map to each allele of a given HLA gene. In certain instances, an individual is determined to be homozygous if the amount of amino acid sequences is at least 2 times or is 5 times more than the next most strongly correlated allele. The application can further comprise a software module determining a full resolution HLA composition, wherein determining the full resolution HLA composition comprises extracting the at least one amino acid sequence read that unambiguously aligns to an individual's 4-digit HLA allele composition and aligning the at least one amino acid sequence read all HLA allele reference sequences that are contained within the 4-digit HLA allele group. In certain instances, the individual's 4-digit HLA allele composition is the major histocompatibility complex (MHC) class I allele composition. In certain instances, the individual's 4-digit HLA allele composition is the major histocompatibility complex (MHC) class II allele composition. In certain instances, the individual's 4-digit HLA allele composition is the major histocompatibility class I and the major histocompatibility class II allele composition. In certain instances, the application results in at least a three-fold reduced runtime by at least three-fold compared with an application running the Optitype method. The application is useful for an individual suffering from an autoimmune disease. The application is useful for an individual in need of an organ transplant.

In another embodiment, described herein, is a computer-implemented system comprising: a digital processing device comprising: at least one processor, an operating system configured to perform executable instructions, a memory, and a computer program including instructions executable by the digital processing device to create an application for determining an individual's 4-digit human leukocyte antigen (HLA) allele composition, the application comprising: a software module mapping at least one nucleic acid sequence read from the individual to known HLA allele reference sequences to identify a first set of HLA alleles that explain the at least one nucleic acid sequence reads; and a software module using a multiple sequence alignment (MSA) of known HLA allele reference sequences to identify one or more additional HLA allele reference sequences that match the at least one nucleic acid sequence read equally well as the first set of HLA alleles; wherein the individual's 4-digit HLA allele composition comprises the one or more additional HLA allele reference sequences that have the closest match to the at least one nucleic acid sequence read from the individual. In certain embodiments, the nucleic acid is DNA. The at least one nucleic acid sequence read can be obtained by a next-generation sequencing technique. The at least one nucleic acid sequence read can be less than 300 nucleotides. The at least one nucleic acid sequence read can be a plurality of nucleic acid sequence reads. The multiple sequence alignment can comprise all known HLA allele reference sequences. The multiple sequence alignment can comprise all known HLA allele reference sequences available from IMGT/HLA database. The first set of HLA alleles can comprise HLA alleles with at least 95% identity to the at least one nucleic acid sequence read from the individual. The one or more additional HLA allele reference sequences can comprise HLA alleles with at least 95% identity to the at least one nucleic acid sequence read from the individual. The application can further comprise a software module generating a solution set and a comparison set, wherein the solution set comprises the one or more additional HLA allele reference sequences that have the closest match to the at least one nucleic acid sequence read from the individual based upon core exons and the comparison set comprises HLA allele reference sequences that performed nearly as well those of the solution set. The core exons can consist of exons 2 and 3 if the HLA allele reference sequence is a class I molecule. The core exons can consist of exon 2 if the HLA allele reference sequence is a class II molecule. The application can further comprise a software module comparing each of the HLA allele reference sequences of the solution set with one or more HLA allele reference sequences of the comparison set based upon all shared exons, wherein the solution set is updated with an HLA allele reference sequence from the comparison set if one or more of the HLA allele reference sequences from the comparison set better explains the at least one nucleic acid sequence read from the individual. The application can be repeated more than once. The application can be repeated until no HLA allele reference sequence from the solution set can be replaced by an HLA allele from the comparison set. In certain instances, only amino acid sequences mapped to an HLA reference sequence of the solution set or to an HLA reference sequence of the comparison set, but not to both, and not to any other HLA allele reference sequence in the solution set are used to evaluate whether the putative HLA allele should be replaced by the comparison allele. The application can be repeated more than once. The application can be repeated until no HLA allele reference sequence from the solution set can be replaced by an HLA allele from the comparison set. In certain instances, the application further comprises checking zygosity, wherein checking zygosity determines whether an individual is heterozygous or homozygous for any one or more HLA alleles of the individual's 4-digit HLA allele composition. Checking zygosity can comprise counting the amino acid sequences that map to each allele of a given HLA gene. In certain instances, the individual is determined to be homozygous if the amount of amino acid sequences is at least 2 times or more than the next most strongly correlated allele. In certain instances, the application further comprises determining a full resolution HLA composition, wherein determining the full resolution HLA composition comprises extracting the at least one amino acid sequence read that unambiguously align to an individual's 4-digit HLA allele composition and aligning the at least one amino acid sequence read all HLA allele reference sequences that are contained within the 4-digit HLA allele group. In certain instances, the individual's 4-digit HLA allele composition is the major histocompatibility complex (MHC) class I allele composition. In certain instances, the individual's 4-digit HLA allele composition is the major histocompatibility complex (MHC) class II allele composition. In certain instances, the individual's 4-digit HLA allele composition is the major histocompatibility class I and the major histocompatibility class II allele composition. In certain instances, the application results in at least a three-fold reduced runtime by at least three-fold compared with an application running the Optitype method. The application is useful for an individual suffering from an autoimmune disease. The application is useful for an individual in need of an organ transplant.

In another embodiment, described herein, is a computer-implemented system comprising: a digital processing device comprising: at least one processor, an operating system configured to perform executable instructions, a memory, and a computer program including instructions executable by the digital processing device to create an application for determining an individual's 4-digit human leukocyte antigen (HLA) allele composition, the application comprising: a software module mapping at least one amino acid sequence translated from at least one nucleic acid sequence read from the individual to known HLA allele reference sequences to identify a first set of HLA alleles that explain the at least one amino acid sequence; and a software module using a multiple sequence alignment (MSA) of known HLA allele reference sequences to identify one or more additional HLA allele reference sequences that match the at least one amino acid sequence equally well as the first set of HLA alleles; wherein the individual's 4-digit HLA allele composition comprises the one or more additional HLA allele reference sequences that have the closest match to the at least one amino acid translated from at least one nucleic acid sequence read from the individual. In certain embodiments, the nucleic acid is DNA. The at least one nucleic acid sequence read can be by a next-generation sequencing technique. The at least one nucleic acid sequence read can be less than 300 nucleotides. The at least one nucleic acid sequence read can be a plurality of nucleic acid sequence reads. The multiple sequence alignment can comprise all known HLA allele reference sequences. The multiple sequence alignment can comprise all known HLA allele reference sequences available from IMGT/HLA database. The first set of HLA alleles can comprise HLA alleles with at least 95% identity to the at least one nucleic acid sequence read from the individual. The one or more additional HLA allele reference sequences can comprise HLA alleles with at least 95% identity to the at least one nucleic acid sequence read from the individual. The application can further comprise a software module generating a solution set and a comparison set, wherein the solution set comprises the one or more additional HLA allele reference sequences that have the closest match to the at least at least one amino acid sequence based upon core exons and the comparison set comprises HLA allele reference sequences that performed nearly as well those of the solution set. The core exons can consist of exons 2 and 3 if the HLA allele reference sequence is a class I molecule. The core exons can consist of exon 2 if the HLA allele reference sequence is a class II molecule. The application can further comprise a software module comparing each of the HLA allele reference sequences of the solution set with one or more HLA allele reference sequences of the comparison set based upon all shared exons, wherein the solution set is updated with an HLA allele reference sequence from the comparison set if one or more of the HLA allele reference sequences from the comparison set better explains the at least one amino acid sequence. The application can be repeated more than once. The application can be run repetitively until no HLA allele reference sequence from the solution set can be replaced by an HLA allele from the comparison set. In some instances, only nucleic acid sequence reads mapped to an HLA reference sequence of the solution set or to an HLA reference sequence of the comparison set, but not to both, and not to any other HLA allele reference sequence in the solution set are used to evaluate whether the putative HLA allele should be replaced by the comparison allele. The application can be repeated more than once. The application can be run repetitively until no HLA allele reference sequence from the solution set can be replaced by an HLA allele from the comparison set. The media of any of claims 79 to 95, wherein the application further comprises a software module checking zygosity, wherein checking zygosity determines whether an individual is heterozygous or homozygous for any one or more HLA alleles of the individual's 4-digit HLA allele composition. Checking zygosity can comprise counting the amino acid sequences that map to each allele of a given HLA gene. In certain instances, an individual is determined to be homozygous if the amount of amino acid sequences is at least 2 times more than the next most strongly correlated allele. The application can further comprise a software module determining a full resolution HLA composition, wherein determining the full resolution HLA composition comprises extracting the at least one amino acid sequence read that unambiguously aligns to an individual's 4-digit HLA allele composition and aligning the at least one amino acid sequence read all HLA allele reference sequences that are contained within the 4-digit HLA allele group. In certain instances, the individual's 4-digit HLA allele composition is the major histocompatibility complex (MHC) class I allele composition. In certain instances, the individual's 4-digit HLA allele composition is the major histocompatibility complex (MHC) class II allele composition. In certain instances, the individual's 4-digit HLA allele composition is the major histocompatibility class I and the major histocompatibility class II allele composition. In certain instances, the application results in at least a three-fold reduced runtime by at least three-fold compared with an application running the Optitype method. The application is useful for an individual suffering from an autoimmune disease. The application is useful for an individual in need of an organ transplant.

In another embodiment, described herein, is a method to refine an HLA allele set from a nucleic acid sequence of an individual comprising comparing each HLA allele reference sequence of a solution set with one or more HLA allele reference sequences of a comparison set based upon all exons shared between the HLA reference sequence of the solution set and an HLA allele reference sequence of the comparison set, wherein the solution set is updated with the HLA allele reference sequence from the comparison set if the HLA allele reference sequence from the comparison set better explains the nucleic acid sequence from the individual. In certain embodiments, the nucleic acid is DNA. The at least one nucleic acid sequence read can be obtained by a next-generation sequencing technique. The at least one nucleic acid sequence read can be less than 300 nucleotides. The at least one nucleic acid sequence read can be a plurality of nucleic acid sequence reads. The solution set can comprise one or more additional HLA allele reference sequences that have the closest match to the nucleic acid sequence from the individual based upon core exons and a comparison set, and the comparison set comprises HLA allele reference sequences that performed nearly as well those of the solution set. The core exons can consist of exons 2 and 3 if the HLA allele reference sequence is a class I molecule. The core exons can consist of exon 2 if the HLA allele reference sequence is a class II molecule. The method can be repeated more than once. The method can be repeated until no HLA allele reference sequence from the solution set can be replaced by an HLA allele from the comparison set. In certain instances, only nucleic acid sequence reads mapped to an HLA reference sequence of the solution set or to an HLA reference sequence of the comparison set, but not to both, and not to any other HLA allele reference sequence in the solution set are used to evaluate whether the putative HLA allele should be replaced by the comparison allele. The method can further comprise checking zygosity, wherein checking zygosity determines whether an individual is heterozygous or homozygous for any one or more HLA alleles of the individual's 4-digit HLA allele composition. Checking zygosity can comprise counting the at least one nucleic acid sequence read that maps to each allele of a given HLA gene. In certain instances, the individual is determined to be homozygous if the amount of sequence reads is at least 2 times or more than the next most strongly correlated allele. In certain instances, the method is performed using a computer and runtime is reduced by at least three-fold compared with a computer running the Optitype method. The method is useful for an individual suffering from an autoimmune disease. The method is useful for an individual in need of an organ transplant.

In another embodiment, described herein, is a method to refine an HLA allele set from an amino acid sequence translated from the nucleic acid sequence of an individual comprising comparing each HLA allele reference sequence of a solution set with one or more HLA allele reference sequences of a comparison set based upon all exons shared between the HLA reference sequence of the solution set and an HLA allele reference sequence of the comparison set, wherein the solution set is updated with the HLA allele reference sequence from the comparison set if the HLA allele reference sequence from the comparison set better explains the nucleic acid sequence reads from the individual. In certain embodiments, the nucleic acid is DNA. The at least one nucleic acid sequence read can be obtained by a next-generation sequencing technique. The at least one nucleic acid sequence read can be less than 300 nucleotides. The at least one nucleic acid sequence read can be a plurality of nucleic acid sequence reads. The solution set can comprise one or more additional HLA allele reference sequences that have the closest match to the amino acid sequence translated from the nucleic acid sequence from the individual based upon core exons and a comparison set, and the comparison set comprises HLA allele reference sequences that performed nearly as well those of the solution set. The core exons can consist of exons 2 and 3 if the HLA allele reference sequence is a class I molecule. The core exons can consist of exon 2 if the HLA allele reference sequence is a class II molecule. The method can be repeated more than once. The method can be repeated until no HLA allele reference sequence from the solution set can be replaced by an HLA allele from the comparison set. In certain instances, only nucleic acid sequence reads mapped to an HLA reference sequence of the solution set or to an HLA reference sequence of the comparison set, but not to both, and not to any other HLA allele reference sequence in the solution set are used to evaluate whether the putative HLA allele should be replaced by the comparison allele. The method can further comprise checking zygosity. Checking zygosity can comprise counting the amino acid sequences that map to each allele of a given HLA gene. In certain instances, the individual is determined to be homozygous if the amount of amino acid sequences is at least 2 times or more than the next most strongly correlated allele. In certain instances, the method is performed using a computer and runtime is reduced by at least three-fold compared with a computer running the Optitype method. The method is useful for an individual suffering from an autoimmune disease. The method is useful for an individual in need of an organ transplant.

In another embodiment, described herein, is a non-transitory computer-readable storage media encoded with a computer program including instructions executable by a processor to create an application for refining an HLA allele set from a nucleic acid sequence of an individual comprising a software module configured to compare each HLA allele reference sequence of a solution set with one or more HLA allele reference sequences of a comparison set based upon all exons shared between the HLA reference sequence of the solution set and an HLA allele reference sequence of the comparison set, wherein the solution set is updated with the HLA allele reference sequence from the comparison set if the HLA allele reference sequence from the comparison set better explains the nucleic acid sequence from the individual. In certain embodiments, the nucleic acid is DNA. The at least one nucleic acid sequence read can be obtained by a next-generation sequencing technique. The at least one nucleic acid sequence read can be less than 300 nucleotides. The at least one nucleic acid sequence read can be a plurality of nucleic acid sequence reads. The solution set can comprise one or more additional HLA allele reference sequences that have the closest match to the nucleic acid sequence from the individual based upon core exons and a comparison set, and the comparison set comprises HLA allele reference sequences that performed nearly as well those of the solution set. The core exons can consist of exons 2 and 3 if the HLA allele reference sequence is a class I molecule. The core exons can consist of exon 2 if the HLA allele reference sequence is a class II molecule. The application can be repeated more than once. The application can be repeated until no HLA allele reference sequence from the solution set can be replaced by an HLA allele from the comparison set. In certain instances, only nucleic acid sequence reads mapped to an HLA reference sequence of the solution set or to an HLA reference sequence of the comparison set, but not to both, and not to any other HLA allele reference sequence in the solution set are used to evaluate whether the putative HLA allele should be replaced by the comparison allele. The application can further comprise a software module configured to check zygosity, wherein checking zygosity determines whether an individual is heterozygous or homozygous for any one or more HLA alleles of the individual's 4-digit HLA allele composition. Checking zygosity can comprise counting the amino acid sequences that map to each allele of a given HLA gene. In certain instances, the individual is determined to be homozygous if the amount of amino acid sequences is at least 2 times or more than the next most strongly correlated allele. In certain instances, the application results in at least a three-fold reduced runtime by at least three-fold compared with an application running the Optitype method. The application is useful for an individual in need of an organ transplant. The application is useful for an individual suffering from an autoimmune disease.

In another embodiment, described herein, is a non-transitory computer-readable storage media encoded with a computer program including instructions executable by a processor to create an application for refining an HLA allele set from an amino acid sequence translated from the nucleic acid sequence of an individual comprising a software module to configured to compare each HLA allele reference sequence of a solution set with one or more HLA allele reference sequences of a comparison set based upon all exons shared between the HLA reference sequence of the solution set and an HLA allele reference sequence of the comparison set, wherein the solution set is updated with the HLA allele reference sequence from the comparison set if the HLA allele reference sequence from the comparison set better explains the nucleic acid sequence reads from the individual. In certain embodiments, the nucleic acid is DNA. The at least one nucleic acid sequence read can be obtained by a next-generation sequencing technique. The at least one nucleic acid sequence read can be less than 300 nucleotides. The at least one nucleic acid sequence read can be a plurality of nucleic acid sequence reads. The solution set can comprise one or more additional HLA allele reference sequences that have the closest match to the amino acid sequence translated from the nucleic acid sequence from the individual based upon core exons and a comparison set, and the comparison set comprises HLA allele reference sequences that performed nearly as well those of the solution set. The core exons can consist of exons 2 and 3 if the HLA allele reference sequence is a class I molecule. The core exons can consist of exon 2 if the HLA allele reference sequence is a class II molecule. The application can be repeated more than once. The application can be repeated until no HLA allele reference sequence from the solution set can be replaced by an HLA allele from the comparison set. In certain instances, only nucleic acid sequence reads mapped to an HLA reference sequence of the solution set or to an HLA reference sequence of the comparison set, but not to both, and not to any other HLA allele reference sequence in the solution set are used to evaluate whether the putative HLA allele should be replaced by the comparison allele. The application can further comprise a software module configured to check zygosity, wherein checking zygosity determines whether an individual is heterozygous or homozygous for any one or more HLA alleles of the individual's 4-digit HLA allele composition. Checking zygosity can comprise counting the amino acid sequences that map to each allele of a given HLA gene. In certain instances, the individual is determined to be homozygous if the amount of amino acid sequences is at least 2 times or more than the next most strongly correlated allele. In certain instances, the application results in at least a three-fold reduced runtime by at least three-fold compared with an application running the Optitype method. The application is useful for an individual suffering from an autoimmune disease. The application is useful for an individual in need of an organ transplant

In another embodiment, described herein, is a computer-implemented system comprising: a digital processing device comprising: at least one processor, an operating system configured to perform executable instructions, a memory, and a computer program including instructions executable by the digital processing device to create an application for refining an HLA allele set from a nucleic acid sequence of an individual comprising a software module to compare each HLA allele reference sequence of a solution set with one or more HLA allele reference sequences of a comparison set based upon all exons shared between the HLA reference sequence of the solution set and an HLA allele reference sequence of the comparison set, wherein the solution set is updated with the HLA allele reference sequence from the comparison set if the HLA allele reference sequence from the comparison set better explains the nucleic acid sequence from the individual. In certain embodiments, the nucleic acid is DNA. The at least one nucleic acid sequence read can be obtained by a next-generation sequencing technique. The at least one nucleic acid sequence read can be less than 300 nucleotides. The at least one nucleic acid sequence read can be a plurality of nucleic acid sequence reads. The solution set can comprise one or more additional HLA allele reference sequences that have the closest match to the nucleic acid sequence from the individual based upon core exons and a comparison set, and the comparison set comprises HLA allele reference sequences that performed nearly as well those of the solution set. The core exons can consist of exons 2 and 3 if the HLA allele reference sequence is a class I molecule. The core exons can consist of exon 2 if the HLA allele reference sequence is a class II molecule. The application can be repeated more than once. The application can be repeated until no HLA allele reference sequence from the solution set can be replaced by an HLA allele from the comparison set. In certain instances, only nucleic acid sequence reads mapped to an HLA reference sequence of the solution set or to an HLA reference sequence of the comparison set, but not to both, and not to any other HLA allele reference sequence in the solution set are used to evaluate whether the putative HLA allele should be replaced by the comparison allele. The application can further comprise a software module to check zygosity, wherein checking zygosity determines whether an individual is heterozygous or homozygous for any one or more HLA alleles of the individual's 4-digit HLA allele composition. Checking zygosity can comprise counting the amino acid sequences that map to each allele of a given HLA gene. In certain instances, the individual is determined to be homozygous if the amount of amino acid sequences is at least 2 times or more than the next most strongly correlated allele. In certain instances, the application results in at least a three-fold reduced runtime by at least three-fold compared with an application running the Optitype method. The application is useful for an individual suffering from an autoimmune disease. The application is useful for an individual in need of an organ transplant

In another embodiment, described herein, is a computer-implemented system comprising: a digital processing device comprising: at least one processor, an operating system configured to perform executable instructions, a memory, and a computer program including instructions executable by the digital processing device to create an application for refining an HLA allele set from an amino acid sequence translated from the nucleic acid sequence of an individual comprising a software module to compare each HLA allele reference sequence of a solution set with one or more HLA allele reference sequences of a comparison set based upon all exons shared between the HLA reference sequence of the solution set and an HLA allele reference sequence of the comparison set, wherein the solution set is updated with the HLA allele reference sequence from the comparison set if the HLA allele reference sequence from the comparison set better explains the nucleic acid sequence reads from the individual. In certain embodiments, the nucleic acid is DNA. The at least one nucleic acid sequence read can be obtained by a next-generation sequencing technique. The at least one nucleic acid sequence read can be less than 300 nucleotides. The at least one nucleic acid sequence read can be a plurality of nucleic acid sequence reads. The solution set can comprise one or more additional HLA allele reference sequences that have the closest match to the amino acid sequence translated from the nucleic acid sequence from the individual based upon core exons and a comparison set, and the comparison set comprises HLA allele reference sequences that performed nearly as well those of the solution set. The core exons can consist of exons 2 and 3 if the HLA allele reference sequence is a class I molecule. The core exons can consist of exon 2 if the HLA allele reference sequence is a class II molecule. The application can be repeated more than once. The application can be repeated until no HLA allele reference sequence from the solution set can be replaced by an HLA allele from the comparison set. In certain instances, only nucleic acid sequence reads mapped to an HLA reference sequence of the solution set or to an HLA reference sequence of the comparison set, but not to both, and not to any other HLA allele reference sequence in the solution set are used to evaluate whether the putative HLA allele should be replaced by the comparison allele. The application can further comprise a software module to check zygosity, wherein checking zygosity determines whether an individual is heterozygous or homozygous for any one or more HLA alleles of the individual's 4-digit HLA allele composition. Checking zygosity can comprise counting the amino acid sequences that map to each allele of a given HLA gene. In certain instances, the individual is determined to be homozygous if the amount of amino acid sequences is at least 2 times or more than the next most strongly correlated allele. In certain instances, the application results in at least a three-fold reduced runtime by at least three-fold compared with an application running the Optitype method. The application is useful for an individual suffering from an autoimmune disease. The application is useful for an individual in need of an organ transplant.

### BRIEF DESCRIPTION OF THE DRAWINGS

A better understanding of the features and advantages of the subject matter described herein will be obtained by reference to the following detailed description that sets forth illustrative embodiments and the accompanying drawings of which:

FIG. 1 illustrates the significant improvements with respect to increased speed, increased accuracy, and reduced computing power needed that are achieved deploying the methods, systems and media of the present disclosure;

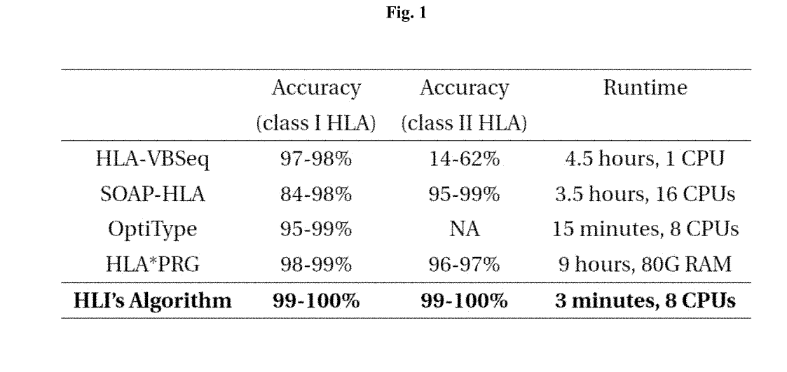

FIG. 2 shows a schematic diagram of the HLA locus;

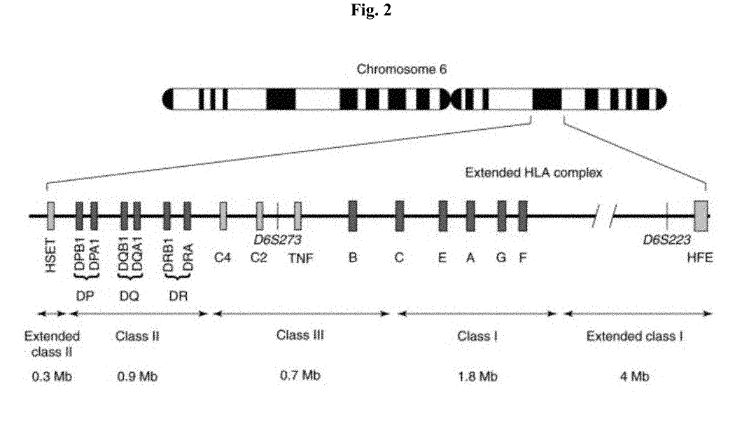

FIG. 3 illustrates the number of alleles for each HLA gene in the IMGT database and exon coverage;

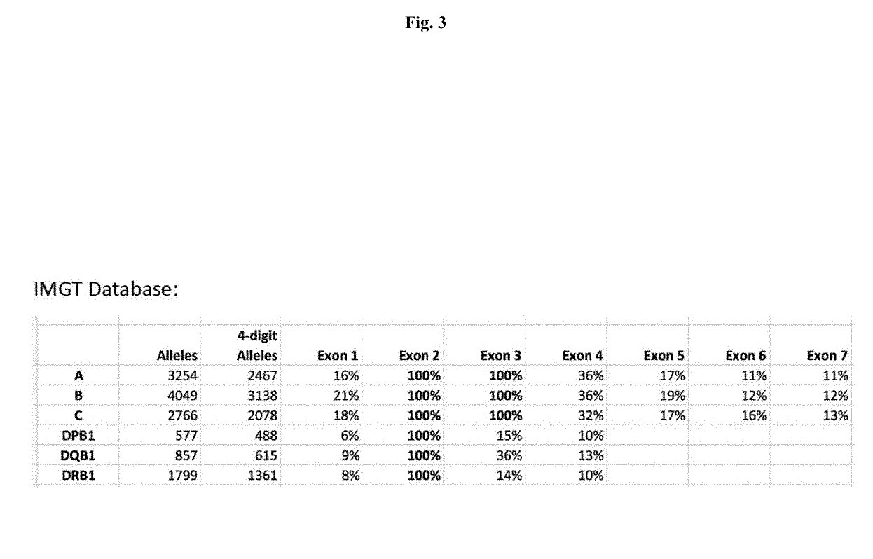

FIG. 4 shows the standard convention for HLA allele nomenclature;

FIG. 5 illustrates the high homology of the different HLA genes in the HLA locus and that individual short reads taken from locus can map to many different HLA alleles and loci;

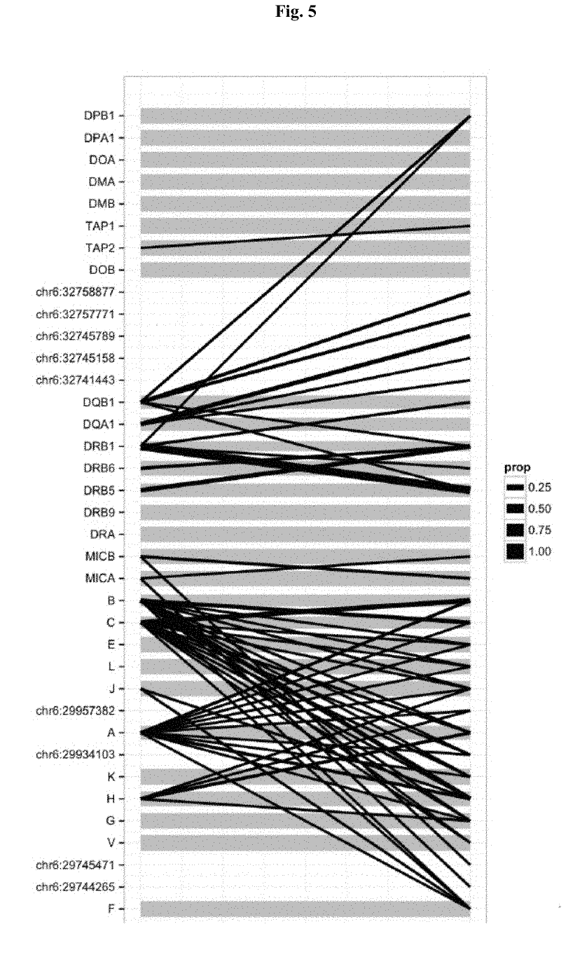

FIG. 6 shows a hypothetical alignment matrix using core exons;

FIG. 7 shows a flow chart depicting the steps executed to determine a full resolution HLA type;

FIG. 8 illustrates a novel feature of the methodology disclosed herein; namely evaluating whether a putative HLA allele explains sequencing data better than a comparison allele, by comparing reads from exons with known reference sequences for both alleles and explainable by only the one of the two alleles but not by other alleles in the current solution set;

FIG. 9 shows a flow chart depicting steps involved iteratively updating the initial solution set with the comparison set;

FIG. 10 shows a flow chart depicting steps involved iteratively updating the solution set with the comparison set;

FIG. 11 shows a schematic illustration of how a given HLA gene is designated as heterozygous or homozygous for a specific allele;

FIG. 12 shows a flow chart depicting steps involved in determining a full resolution HLA type; and

FIG. 13 shows a non-limiting example of a digital processing device; in this case, a device with one or more CPUs, a memory, a communication interface, and a display. The devices and connectivity can be used to deliver reports accessible by health care professionals. The reports can be generated by any of the methods of the current disclosure.

### DETAILED DESCRIPTION OF THE INVENTION

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

As used herein “reference genome” refers to any standard publicly available reference genome, for example GRCh38, the Genome Reference Consortium human genome (build 38). Alternatively, the reference genome can be one that is constructed de novo from sequencing a plurality of genomes. In certain embodiments, the plurality of genomes is greater than 10,000 different genomes. In certain embodiments, the plurality of genomes is greater than 100,000 different genomes.

The methods, systems and media of this disclosure represent a substantial improvement on current HLA typing methods. The method described herein uses nucleic acid sequence reads generated from an individual's genome. In certain embodiments, the nucleic acid sequence is DNA. The nucleic acid sequence reads can be generated using any nucleic acid sequencing technology, but the full power of the method is realized using short reads generated using next-generation sequencing technologies. The technology can be any next generation technology that generates short reads such as pyrosequencing, sequencing by synthesis, sequencing by ligation, ion semiconductor sequencing, and/or sequencing arrays. The method is also compatible with older sequencing technologies such as Sanger sequencing. The reads can be paired-end reads. The average length of a nucleic acid sequence read can be less than 500, 400, 300, 200, 150, 100, 75, 50, 40, 35, 32 or 30 base pairs. Any number of reads can be used, in some cases a plurality of reads are used. In some cases, 10, 20, 30, 40, 50, 60, 70, 80, 90, 100, 500, 1,000, 2,000 or more reads are used, including increments therein.

The nucleic acid reads can be derived from DNA or RNA, isolated from a biological sample such as blood, plasma, serum, biopsy, saliva, urine or semen. The nucleic acid reads can also be cDNA reverse transcribed from the nucleic acids isolated from abiological sample. In a certain aspect, the nucleic acid can be a circulating cell free DNA or RNA. In certain instances, the DNA analyzed is nuclear genomic DNA and not mitochondrial DNA. Nucleic acid sequences from an individual can be obtained by a third-party sequencing provider or a previously determined sequence that an individual may transmit to a facility or individual performing the method herein. The individual can be a patient that is receiving a transplant or is on a transplant list, or is a prospective organ donor. In a certain embodiment, the sequencing methods used herein can be useful for prognosing or diagnosing an autoimmune disease.

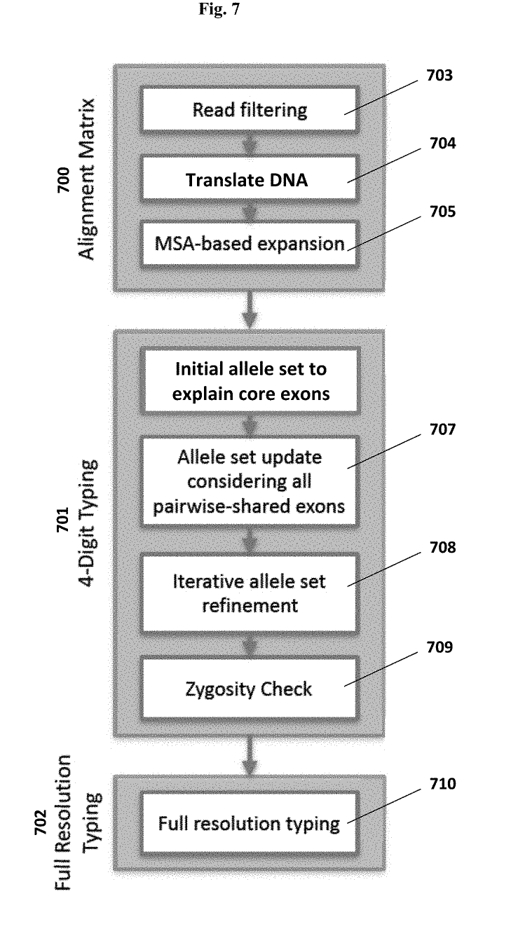

FIG. 7 shows a schematic with a flow-chart depicting the overall methodology. In a first step 700, one or more independent nucleic acid sequence reads are aligned to the HLA locus to make an alignment matrix representing which sequence reads mapped to which known HLA alleles. Raw sequencing data are filtered 703. This filtering step removes low quality base pairs from the ends (trimming) and rejects sequence reads with overall low quality. The reads are then aligned to known HLA alleles. In some instances, reads that have a minimum of 100 base pairs are preferred. In some instances, reads that have a minimum of 50 base pairs can be used. In an alternative embodiment 704, the nucleic acid is translated to an amino acid sequence before aligning to the amino acid sequences of known HLA alleles. This novel step reduces the overall matching to 4-digit alleles, and, thus, only matches to the genes that are most important for HLA typing in the clinic. It also allows for faster alignments since every non-synonymous SNP is of greater impact than 10 synonymous SNPs. After reads have been filtered, reads are aligned to known HLA alleles to identify a first set of HLA alleles that share common sequences with the sequence reads. Due to the large number of similar but different alleles for each HLA gene, the initial alignment will often miss some matches that are equally as good, or nearly as good, as the first set of HLA alleles. Therefore, this first set of HLA alleles is then expanded using a multiple sequence alignment 705 of known HLA alleles, resulting in one or more additional HLA alleles being identified that share common sequences with the sequence reads. In some cases, the MSA based expansion uses all known HLA sequences. The effect of this step is to create an exhaustive list of HLA alleles that could be explained by the reads. In some cases, the first set of HLA alleles comprises alleles that are 100% identical to any sequence read. In some cases, the first set of HLA alleles comprises alleles that are at least 99%, 98, 97%, 96%, or 95% identical to any sequence read. In some cases, the expanded list of HLA alleles comprises alleles that are 100% identical to any allele in the first set. In some cases, the expanded list of HLA alleles comprises alleles that are at least 99%, 98, 97%, 96%, or 95% identical to any allele in the first set.

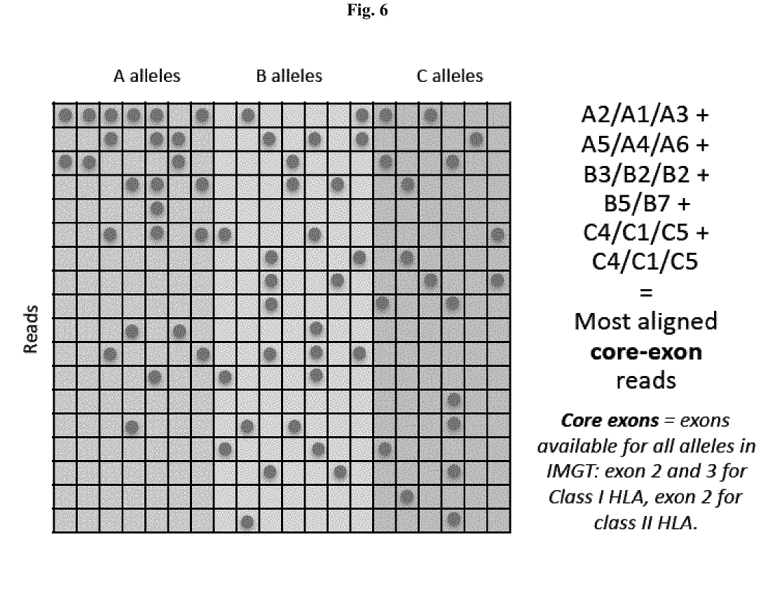

The alignment matrix generated by step 700 can be analyzed to find the individual's 4-digit HLA composition 701 by determining the top two alleles for each gene that are explained by the expanded HLA allele set 706. In a certain, embodiment, the top two alleles are based on a ranking or a probability metric. This step may be performed by using core exons and the integer linear programming method. This can be done using an alignment matrix as depicted in FIG. 6. Core exons are exons 2 and 3 for class I, or exon 2 for class II HLA. Additionally, as described in this disclosure, a zygosity check 709 can be performed to determine if an individual is heterozygous or homozygous for a specified allele at a given locus.

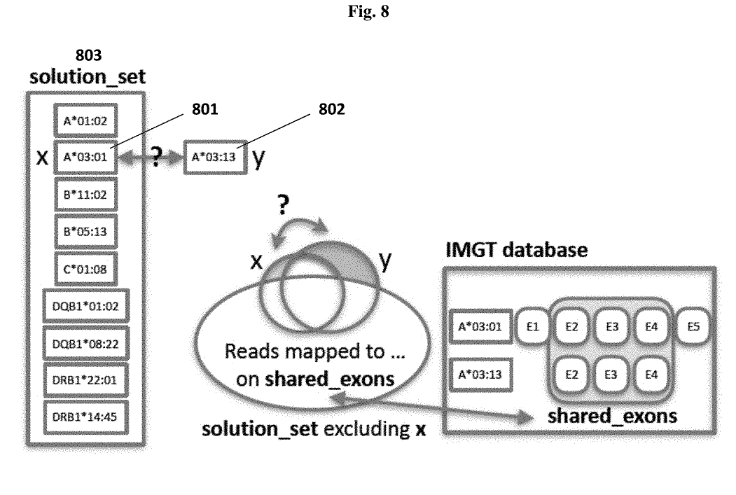

FIG. 8 illustrates that the accuracy of the results from step 701 can be further improved by expanding the analysis from core exons to all available shared exons from the expanded allele set. For example, two alleles 801 and 802 may be equally or nearly equally able to be included in an initial solution set 803 that contains a plurality of HLA alleles that best explain an individual's nucleic acid or translated amino acid sequence reads. In this case expanding the analysis to exons shared between two different alleles can increase the overall confidence of a solution set or resolve ambiguity. Allele 802 may be substituted for allele 801 in the solution set, and if the inclusion of 802 creates a solution set that better explains an individual's sequence reads then 802 is retained in the set; if not 801 is retained. A solution set better explains an individual's sequence reads if it has a higher confidence in the alignment. This can take into account sequencing quality, error probability or both. In a certain embodiment, a sequencing quality greater than 10%, 20, 30%, 40%, 50%, 60%, 70%, 80%, 90% or higher will result in higher confidence. In a certain embodiment, an error probability that is 1.5-fold, 2-fold, 3-fold, 4-fold, 5-fold, 6-fold, 7-fold, 8-fold, 9-fold, 10-fold or lower will result in higher confidence. Alternatively, if more shared reads map to allele 802, than allele 802 may be included in the solution set over allele 801. In some cases, only sequence reads explainable by one of the two alleles being compared but not by any other alleles in the current solution set is used in above procedure.

Step 701 can be further improved by iteratively updating alleles from the expanded allele set that best explain the reads 708. In some embodiments, the iterative updating considers all pairwise shared exons 707. In some embodiments, the iterative updating is first performed using core exons. Then, if desired, the iterative process can be repeated using shared exons in addition to core exons.

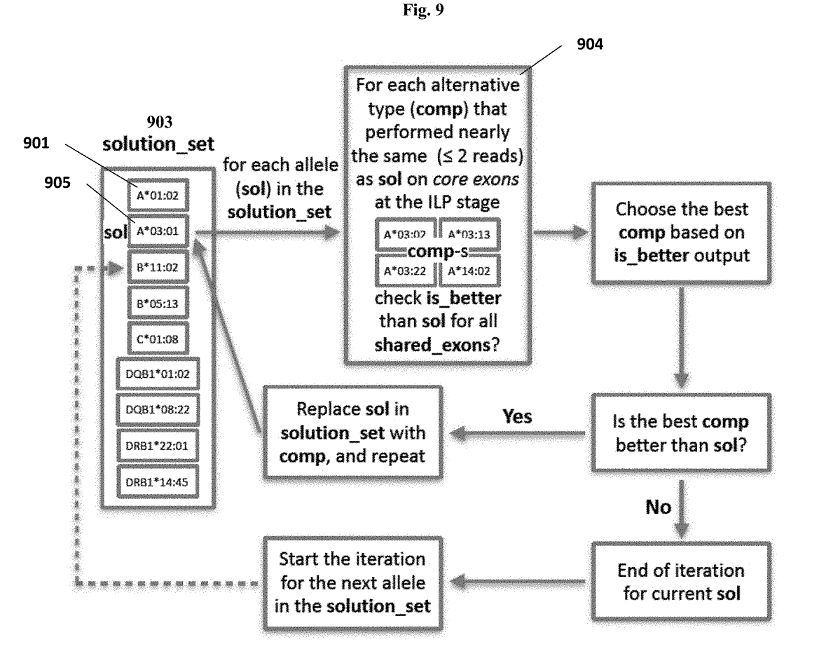

FIG. 9 represents a scheme whereby an initial solution set 903 is generated using sequence reads that map to core exons, and a comparison set 904 is generated using alleles that performed nearly as well as the initial solution set with respect to matching of the individual's sequence reads. In certain instances, an allele performs nearly as well based on ranking. For example, the top ranked allele may be in the initial set and a comparison set comprises alleles ranked 2^{nd}, 3^{rd}, 4^{th}, 5^{th}, 6^{th}, 7^{th}, 8^{th}, 9^{th}, 10^{th} or better; or in the top 1%, 2%, 3%, 4% 5%, or 10% of ranked results. Much as in FIG. 8, the analysis is expanded to shared exons, except that each allele in the solution set is iteratively updated making multiple successive pair-wise comparisons with all alleles in the comparison set. This iterative process can depend on the exact number and coverage of the reads. In certain instances the iterative process continues until no better matching set of HLA alleles is found. In certain instances, this updating goes through at least 1, 2, 3, 4, 5, 6, 7, 8, 9, 10 or more iterations. In certain instances, this updating goes through at least 10, 20, 30, 40, 50, 60, 70, 80, 90, 100 or more iterations, including increments therein. In certain instances, this updating goes through at least 1,000; 2,000; 3,000; 4,000; 5,000; 6,000; 7,000; 8,000; 9,000; 10,000 or more iterations, including increments therein. In certain instances, this updating goes through at least 10, 20, 30, 40, 50, 60, 70, 80, 90, 100 or more iterations, including increments therein. When all combinations are exhausted for the first allele 901 in the solution set then the method moves on to the next allele 905 in the solution set. This is repeated until all alleles in the solution set have been compared to all alleles in the comparison set.

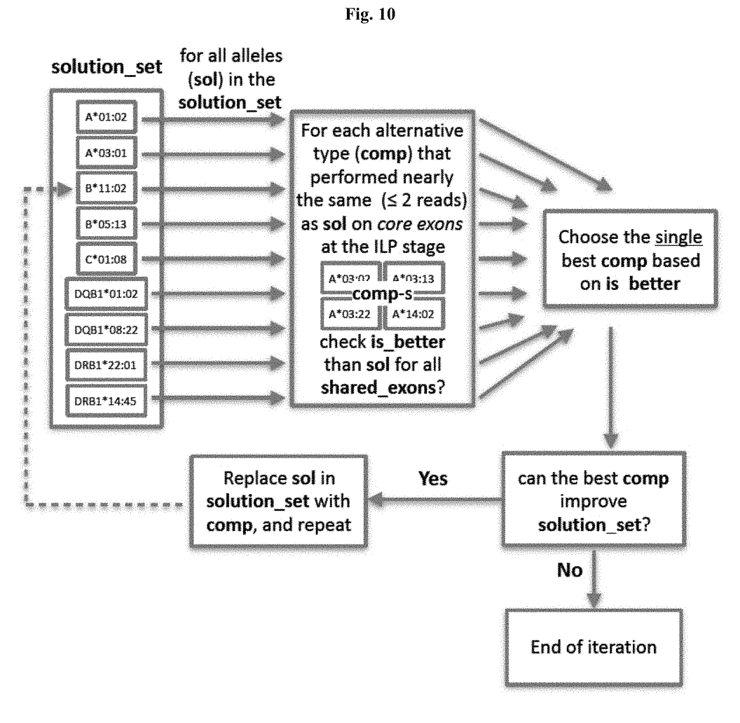

FIG. 10 illustrates that the single best comparison set is chosen to arrive at the individual's 4-digit HLA type. This is achieved by iteratively updating the solution set as a whole, much as in FIG. 9 where individual allele was updated rather than the solution set as a whole.

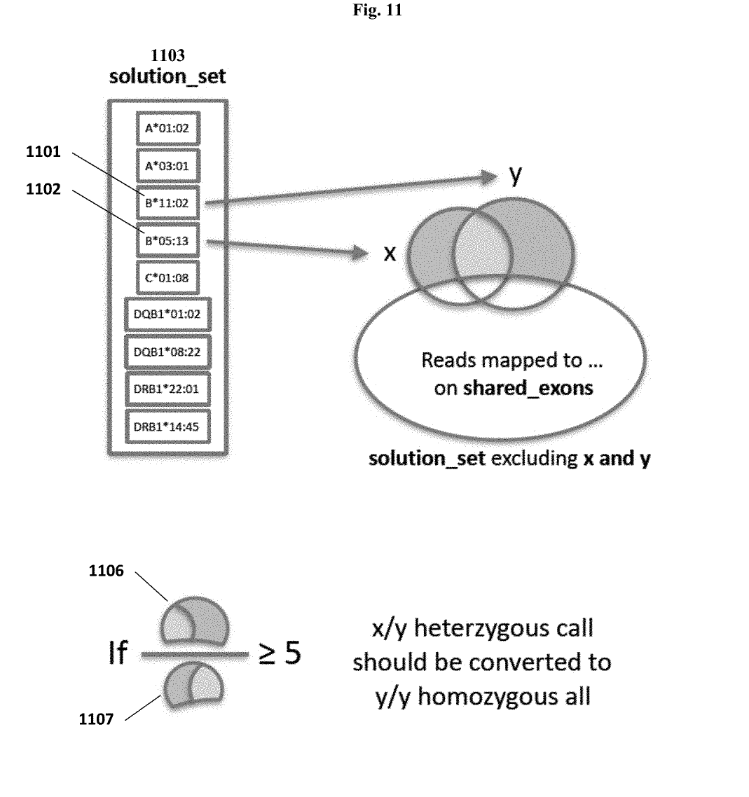

Since the HLA locus is on an autosome, an individual possess two copies of each HLA gene. The individual can be either homozygous or heterozygous at a given HLA gene. FIG. 11 illustrates the concept between a zygosity check. Since the initial solution set will have two alleles for each gene, the final solution set 1103 will have two alleles 1101 and 1102 for each gene. A determination of whether the individual is heterozygous or homozygous for either of alleles 1101 or 1102 one involves counting reads that map to each allele and taking the ratio of reads from one allele to another. In some cases, only reads that can only be explained by one of the two alleles but not by any other allele in the solution set are used. If the number of reads 1106 that map to allele 1101 is sufficiently larger than the number of reads 1107 that map to allele, 1102 there is a high certainty that the individual is homozygous for allele 1101. The ratio of reads that determines a homozygous call can be at least 2:1, 3:1, 4:1, 5:1, 6:1, or more. In certain instances, the allele that is called homozygous has 2, 3, 4, 5, 6, or more times as many reads mapped to it than the next closest allele.

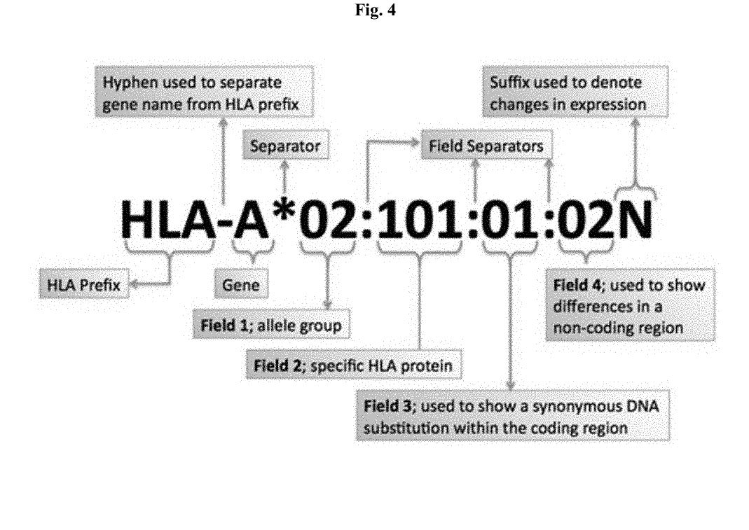

In addition to determining an individual's 4-digit HLA type, the methods of this disclosure allow the determination of a full resolution HLA type. A full resolution HLA type is the individuals HLA composition signified by each allele being identified to all four field codes as represented in FIG. 4.

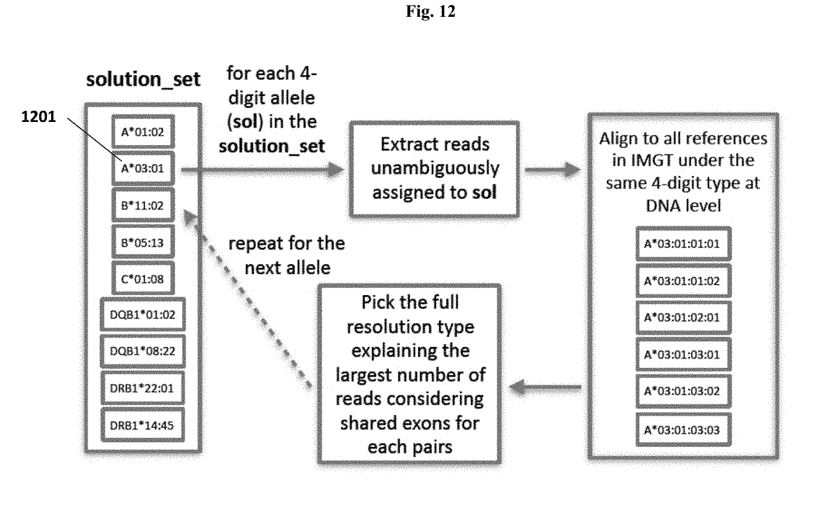

FIG. 12 illustrates a schematic for determining an individual's full resolution HLA type. After a final 4-digit HLA composition is arrived at, and a zygosity check is performed then all reads that are unambiguously assigned to the solution set are aligned to all alleles that fall within each 4-digit type. For example, in FIG. 12 reads that unambiguously map to allele 1201 designated by its 4-digit type A*03:01 are extracted and tested against all other alleles that are of this same 4-digit type A*03:01:01:01, A*03:01:01:02, A*03:01:02:01, A*03:01:03:01, A*03:01:03:02, and A*03:01:03:03. The single best match is added to the solution set, and the process is repeated for all the alleles in the final 4-digit HLA composition set.

### Digital Processing Device

The systems, media, and methods described herein may include a digital processing device, or use of the same. The digital processing device includes one or more hardware central processing units (CPUs) or general purpose graphics processing units (GPGPUs) that carry out the device's functions. The digital processing device further comprises an operating system configured to perform executable instructions. The digital processing device may be reversibly connected a computer network. In various embodiments, the digital processing device is optionally and reversibly connected to: the Internet such that it accesses the World Wide Web, a cloud computing infrastructure, an intranet, and/or a data storage device.

In accordance with the description herein, suitable digital processing devices include, by way of non-limiting examples, server computers, desktop computers, laptop computers, notebook computers, sub-notebook computers, netbook computers, netpad computers, handheld computers, Internet appliances, mobile smartphones, and tablet computers. Those of skill in the art will recognize that many smartphones are suitable for use in the system described herein. Suitable tablet computers include those with booklet, slate, and convertible configurations, known to those of skill in the art.

The digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

The digital processing device includes a storage and/or memory device. The storage and/or memory device is one or more physical apparatuses used to store data or programs on a temporary or permanent basis. In some embodiments, the device is volatile memory and requires power to maintain stored information. In some cases, the memory device is non-volatile memory and retains stored information when the digital processing device is not powered. In various embodiments, the non-volatile memory comprises: flash memory, dynamic random-access memory (DRAM), ferroelectric random access memory (FRAM), and/or phase-change random access memory (PRAM). In other cases, the memory device is a storage device including, by way of non-limiting examples, CD-ROMs, DVDs, flash memory devices, magnetic disk drives, magnetic tapes drives, optical disk drives, and cloud computing based storage. The storage and/or memory device may be a combination of memory devices such as those disclosed herein.

The digital processing device optionally includes a display to send visual information to a user. Many types of display are suitable including, by way of examples, liquid crystal displays (LCD), thin film transistor liquid crystal displays (TFT-LCD), organic light emitting diode (OLED) displays (including passive-matrix OLED (PMOLED) and/or active-matrix OLED (AMOLED) displays), and plasma displays. In some cases, the display is a touchscreen or multi-touchscreen display. Other suitable displays include video projectors and head-mounted displays in communication with the digital processing device, such as a VR headset. Suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. The display may be one or more displays and include a combination of devices such as those disclosed herein.

The digital processing device optionally includes an input device to receive information from a user. In various embodiments, the input device is: a keyboard, a pointing device including, by way of non-limiting examples, a mouse, trackball, track pad, joystick, game controller, or stylus, a touch screen or a multi-touch screen, a microphone to capture voice or other sound input, and/or a video camera or other sensor to capture motion or visual input. In a particular embodiment, the input device is a Kinect, Leap Motion, or the like. The input device may a combination of devices such as those disclosed herein.

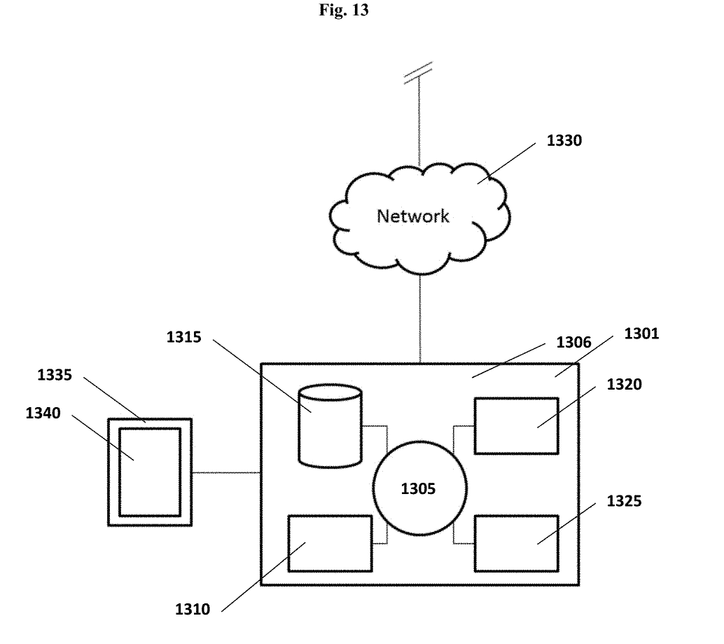

Referring to FIG. 13, in a particular embodiment, an exemplary digital processing device 1301 is programmed or otherwise configured to execute the programs described herein. In this embodiment, the digital processing device 1301 includes a central processing unit (CPU, also “processor” and “computer processor” herein) 1305, which can be a single core or multi core processor, or a plurality of processors for parallel processing. The digital processing device 1301 also includes memory or memory location 1310 (e.g., random-access memory, read-only memory, flash memory), electronic storage unit 1315 (e.g., hard disk), communication interface 1320 (e.g., network adapter) for communicating with one or more other systems, and peripheral devices 1325, such as cache, other memory, data storage and/or electronic display adapters. The memory 1810, storage unit 1315, interface 1320 and peripheral devices 1325 are in communication with the CPU 1305 through a communication bus (solid lines), such as a motherboard. The storage unit 1315 can be a data storage unit (or data repository) for storing data. The digital processing device 1301 can be operatively coupled to a computer network (“network”) 1330 with the aid of the communication interface 1320. The network 1330 can be the Internet, an internet and/or extranet, or an intranet and/or extranet that is in communication with the Internet. The network 1330 in some cases is a telecommunication and/or data network. The network 1330 can include one or more computer servers, which can enable distributed computing, such as cloud computing. The network 1330, in some cases with the aid of the device 1301, can implement a peer-to-peer network, which may enable devices coupled to the device 1301 to behave as a client or a server.

Continuing to refer to FIG. 13, the CPU 1305 can execute a sequence of machine-readable instructions, which can be embodied in a program or software. The instructions may be stored in a memory location, such as the memory 1310. The instructions can be directed to the CPU 1305, which can subsequently program or otherwise configure the CPU 1305 to implement methods of the present disclosure. Examples of operations performed by the CPU 1305 can include fetch, decode, execute, and write back. The CPU 1305 can be part of a circuit, such as an integrated circuit. One or more other components of the device 1301 can be included in the circuit. In some cases, the circuit is an application specific integrated circuit (ASIC) or a field programmable gate array (FPGA).

Continuing to refer to FIG. 13, the storage unit 1315 can store files, such as drivers, libraries and saved programs. The storage unit 1315 can store user data, e.g., user preferences and user programs. The digital processing device 1301 in some cases can include one or more additional data storage units that are external, such as located on a remote server that is in communication through an intranet or the Internet.

Continuing to refer to FIG. 13, the digital processing device 1301 can communicate with one or more remote computer systems through the network 1330. For instance, the device 1301 can communicate with a remote computer system of a user. Examples of remote computer systems include personal computers (e.g., portable PC), slate or tablet PCs (e.g., Apple® iPad, Samsung® Galaxy Tab), telephones, or smart phones (e.g., Apple® iPhone, Android-enabled device, Blackberry®).

Methods as described herein can be implemented by way of machine (e.g., computer processor) executable code stored on an electronic storage location of the digital processing device 1301, such as, for example, on the memory 1310 or electronic storage unit 1315. The machine executable or machine-readable code can be provided in the form of software. During use, the code can be executed by the processor 1305. In some cases, the code can be retrieved from the storage unit 1315 and stored on the memory 1310 for ready access by the processor 1305. In some situations, the electronic storage unit 1315 can be precluded, and machine-executable instructions are stored on memory 1310.

Reports can be delivered from for example a sequencing lab to a health care provider or consumer over the network 1330, or alternatively through the mail or a secure download site such as an FTP site.

### Short Read Sequence Alignement Methods and Software

Any suitable alignment method or software can be used to align short reads described in this disclosre including any one or more of BarraCUDA, BBMap, BFAST, BigBWA, BLASTN, BLAT, Bowtie, HIVE-hexagon, BWA, BWA-PSSM, CASHX, Cloudburst, CUDA-EC, CUSHAW, CUSHAW2, CUSHAW2-GPU, CUSHAW3, drFAST, ELAND, ERNE, GASSST, GEM, Genalice MAP, Geneious Assembler, GensearchNGS, GMAP and GSNAP, GNUMAP, ISAAC, LAST, MAQ, mrFAST, mrsFAST, MOM, MOSAIK, MPscan, Novoalign & NovoalignCS, NextGENe, NextGenMap, Omixon Variant Toolkit, PALMapper, Partek Flow, PASS, PerM, PRIMEX, QPalma, RazerS, REAL, cREAL, RMAP, rNA, RTG Investigator, Segemehl, SeqMap, Shrec, SHRiMP, SLIDER, SOAP, SOAP2, SOAP3, SOAP3-dp, SOCS, SparkBWA, SSAHA, SSAHA2, Stampy, SToRM, Subread, Subjunc, Taipan, UGENE, VelociMapper, XpressAlign, or ZOOM.

### Non-Transitory Computer Readable Storage Medium

The systems, media, and methods disclosed herein may include one or more non-transitory computer readable storage media encoded with a program including instructions executable by the operating system of an optionally networked digital processing device. The computer readable storage medium may be a tangible component of the digital processing device, which may be optionally removable from the digital processing device. Many types of media are suitable to store the instructions. In various embodiments, suitable computer readable storage medium include, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

The systems, media, and methods disclosed herein may include one or more computer programs, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some cases, a computer program comprises one sequence of instructions. In other cases, a computer program comprises a plurality of sequences of instructions. In some cases, a computer program is provided from one location. In other cases, a computer program is provided from a plurality of locations. In various embodiments, a computer program includes, in part or in whole, one or more software modules, one or more web applications, one or more mobile applications, one or more standalone applications, one or more web browser plug-ins, extensions, add-ins, or add-ons, or combinations thereof.

### Standalone Application

A computer program may comprise a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™ Lisp, Python™, Visual Basic, and VB.NET, or combinations thereof. Compilation is often performed, at least in part, to create an executable program. In some cases, a computer program includes one or more executable complied applications.

### Software Modules

The systems, media, and methods disclosed herein may include one or more software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

The systems, media, and methods disclosed herein may include one or more databases, or use of the same. In view of the disclosure provided herein, those of skill in the art will recognize that many databases are suitable for storage and retrieval of nucleic acid and amino acid sequences including HLA allele reference sequences. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### Examples

The following illustrative examples are representative of embodiments of the software applications, systems, and methods described herein and are not meant to be limiting in any way.

### Example 1—HLA Typing of Prospective Organ Transplant Recipient

In this example a patient who is diagnosed with end-stage renal disease will have their 4-digit HLA type determined so that he can be matched with a prospective donor. The patient provides a blood sample sent to a CLIA compliant facility from which DNA is extracted and sequenced using a next-generation sequencing technology such as the MiSeg™ or HiSeg™ system available from Illumina, Inc. The sequencing results will be analyzed at the facility using the methods of this discloser and the 4-digit-HLA type is transmitted to a health care service provider. At the same time an individual who may be a prospective donor, in this case a sibling of the patient, will have their 4-digit HLA type determined in the same way. Alternatively, the raw sequencing data can be transmitted to the health care provider for analysis and HLA determination.

### Example 2—HLA Typing to Determine Type 1 Diabetes Risk

In this example a healthy-individual is tested to determine a risk of developing Type 1 diabetes. The individual provides a saliva sample sent to a CLIA compliant facility from which DNA is extracted and sequenced using a next-generation sequencing technology such as the MiSeg™ or HiSeg™ system available from Illumina, Inc. The sequencing results will be analyzed at the facility using the methods of this discloser and the 4-digit-HLA type is transmitted to a health care service provider. If the individual's HLA haplotype is a haplotype that is particularly associated with a high risk for developing type I diabetes (e.g., DRB1*03:01-DQA1*05:01-DQB1*02:01 or DRB1*04:01/02/04/05/08-DQA1*03:01-DQB1*03:02/04) then the individual will be monitored more closely in the future for early stage type I diabetes.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

What is claimed is:

1. A method of determining an individual's 4-digit human leukocyte antigen (HLA) allele composition, the method comprising:
a) mapping at least one nucleic acid sequence read from the individual to known HLA allele reference sequences to identify a first set of HLA alleles that explain the at least one nucleic acid sequence read; and
b) using a multiple sequence alignment (MSA) of known HLA allele reference sequences to identify one or more additional HLA allele reference sequences that match the at least one nucleic acid sequence read equally well as the first set of HLA alleles;
wherein the individual's 4-digit HLA allele composition comprises the one or more additional HLA allele reference sequences that have the closest match to the at least one nucleic acid sequence read from the individual.

2. The method of claim 1, wherein nucleic acid sequence read is a DNA sequence read.

3. The method of claim 1, wherein the at least one nucleic acid sequence read is obtained by a next-generation sequencing technique.

4. The method of claim 1, wherein the at least one nucleic acid sequence read is less than 300 nucleotides.

5. The method of claim 1, wherein the at least one nucleic acid sequence read is a plurality of nucleic acid sequence reads.

6. The method of claim 1, wherein the multiple sequence alignment comprises all known HLA allele reference sequences.

7. The method of claim 1, wherein the multiple sequence alignment comprises all known HLA allele reference sequences available from IMGT/HLA database.

8. The method of claim 1, wherein the first set of HLA alleles comprise HLA alleles with at least 95% identity to the at least one nucleic acid sequence read from the individual.

9. The method of claim 1, wherein the one or more additional HLA allele reference sequences comprise HLA alleles with at least 95% identity to the at least one nucleic acid sequence read from the individual.

10. The method of claim 1, further comprising generating a solution set and a comparison set, wherein the solution set comprises the one or more additional HLA allele reference sequences that have the closest match to the at least one nucleic acid sequence read from the individual based upon core exons and the comparison set comprises HLA allele reference sequences that performed nearly as well those of the solution set.

11. The method of claim 10, wherein the core exons consist of exons 2 and 3 if the HLA allele reference sequence is a class I molecule.

12. The method of claim 10, wherein the core exons consist of exon 2 if the HLA allele reference sequence is a class II molecule.

13. The method of claim 10, further comprising comparing each of the HLA allele reference sequences of the solution set with one or more HLA allele reference sequences of the comparison set based upon all shared exons, wherein the solution set is updated with an HLA allele reference sequence from the comparison set if one or more of the HLA allele reference sequences from the comparison set better explains the nucleic acid sequence reads from the individual.

14. The method of claim 13, repeated more than once.

15. The method of claim 13, repeated until no HLA allele reference sequence from the solution set can be replaced by an HLA allele from the comparison set.

16. The method of claim 13, wherein only nucleic acid sequence reads mapped to an HLA reference sequence of the solution set or to an HLA reference sequence of the comparison set, but not to both, and not to any other HLA allele reference sequence in the solution set are used to evaluate whether the putative HLA allele should be replaced by the comparison allele.

17. The method of claim 16, repeated more than once.

18. The method of claim 16, repeated until no HLA allele reference sequence from the solution set can be replaced by an HLA allele from the comparison set.

19. The method of claim 18, further comprising checking zygosity, wherein checking zygosity determines whether an individual is heterozygous or homozygous for any one or more HLA alleles of the individual's 4-digit HLA allele composition.

20. The method of claim 19, wherein checking zygosity comprises counting the at least one nucleic acid sequence read that maps to each allele of a given HLA gene.

21. The method of claim 20, wherein the individual is determined to be homozygous if the amount of sequence reads is at least 2 times or more than the next most strongly correlated allele.

22. The method of any of claim 19, further comprising determining a full resolution HLA composition, wherein determining the full resolution HLA composition comprises extracting the at least one nucleic acid sequence read that unambiguously align to an individual's 4-digit HLA allele composition and aligning the at least one nucleic acid sequence read to all HLA allele reference sequences that are contained within the 4-digit HLA allele group.

23. The method of claim 1, wherein the individual's 4-digit HLA allele composition is the major histocompatibility complex (MHC) class I allele composition.

24. The method of claim 1, wherein the individual's 4-digit HLA allele composition is the major histocompatibility complex (MHC) class II allele composition.

25. The method of claim 1, wherein the individual's 4-digit HLA allele composition is the major histocompatibility class I and the major histocompatibility class II allele composition.

26. The method of claim 1, wherein the method is performed using a computer wherein the runtime is reduced by at least three-fold compared with a computer running the Optitype method.

27. The method of claim 1, wherein the individual suffers from an autoimmune disease.

28. The method of any of claim 1, wherein the individual is in need of an organ transplant.

29. A method of determining an individual's 4-digit human leukocyte antigen (HLA) allele composition, the method comprising:
a) mapping at least one amino acid sequence translated from at least one nucleic acid sequence read from the individual to known HLA allele reference sequences to identify a first set of HLA alleles that explain the at least one amino acid sequence; and
b) using a multiple sequence alignment (MSA) of known HLA allele reference sequences to identify one or more additional HLA allele reference sequences that match the at least one amino acid sequence equally well as the first set of HLA alleles;
wherein the individual's 4-digit HLA allele composition comprises the one or more additional HLA allele reference sequences that have the closest match to the at least one amino acid translated from at least one nucleic acid sequence read from the individual.

30. The method of claim 29, wherein nucleic acid sequence read is a DNA sequence read.

31. The method of claim 29, wherein the at least one nucleic acid sequence read is by a next-generation sequencing technique.

32. The method of claim 29, wherein the at least one nucleic acid sequence read is less than 300 nucleotides.

33. The method of claim 29, wherein the at least one nucleic acid sequence read is a plurality of nucleic acid sequence reads.

34. The method of claim 29, wherein the multiple sequence alignment comprises all known HLA allele reference sequences.

35. The method of claim 29, wherein the multiple sequence alignment comprises all known HLA allele reference sequences available from IMGT/HLA database.

36. The method of claim 29, wherein the first set of HLA alleles comprise HLA alleles with at least 95% identity to the at least one amino acid sequence translated from at least one nucleic acid sequence read from the individual.

37. The method of claim 29, wherein the one or more additional HLA allele reference sequences comprise HLA alleles with at least 95% identity to the at least one amino acid sequence translated from at least one nucleic acid sequence read from the individual.

38. The method of claim 29, further comprising generating a solution set and a comparison set, wherein the solution set comprises the one or more additional HLA allele reference sequences that have the closest match to the at least at least one amino acid sequence based upon core exons and the comparison set comprises HLA allele reference sequences that performed nearly as well those of the solution set.

39. The method of claim 38, wherein the core exons consist of exons 2 and 3 if the HLA allele reference sequence is a class I molecule.

40. The method of claim 38, wherein the core exons consist of exon 2 if the HLA allele reference sequence is a class II molecule.

41. The method of claim 38, further comprising comparing each of the HLA allele reference sequences of the solution set with one or more HLA allele reference sequences of the comparison set based upon all shared exons, wherein the solution set is updated with an HLA allele reference sequence from the comparison set if one or more of the HLA allele reference sequences from the comparison set better explain the sequence data from the individual.

42. The method of claim 41, repeated more than once.

43. The method of claim 41, repeated until no HLA allele reference sequence from the solution set can be replaced by an HLA allele from the comparison set.

44. The method of claim 41, wherein only amino acid sequences mapped to an HLA reference sequence of the solution set or to an HLA reference sequence of the comparison set, but not to both, and not to any other HLA allele reference sequence in the solution set are used to evaluate whether the putative HLA allele should be replaced by the comparison allele.

45. The method of claim 44, repeated more than once.

46. The method of claim 44, repeated until no HLA allele reference sequence from the solution set can be replaced by an HLA allele from the comparison set.

47. The method of claim 41, further comprising checking zygosity, wherein checking zygosity determines whether an individual is heterozygous or homozygous for any one or more HLA alleles of the individual's 4-digit HLA allele composition.

48. The method of claim 47, wherein checking zygosity comprises counting the amino acid sequences that map to each allele of a given HLA gene.

49. The method of claim 47, wherein the individual is determined to be homozygous if the amount of amino acid sequences is at least 2 times or more than the next most strongly correlated allele.

50. The method of claim 47, further comprising determining a full resolution HLA composition, wherein determining the full resolution HLA composition comprises extracting the at least one amino acid sequence read that unambiguously align to an individual's 4-digit HLA allele composition and aligning the at least one amino acid sequence read all HLA allele reference sequences that are contained within the 4-digit HLA allele group.

51. The method of claim 29, wherein the individual's 4-digit HLA allele composition is the major histocompatibility complex (MEW) class I allele composition.

52. The method of claim 29, wherein the individual's 4-digit HLA allele composition is the major histocompatibility complex (MEW) class II allele composition.

53. The method of claim 29, wherein the individual's 4-digit HLA allele composition is the major histocompatibility class I and the major histocompatibility class II allele composition.

54. The method of claim 29, wherein the method is performed using a computer wherein the runtime is reduced by at least three-fold compared with a computer running the Optitype method.

55. The method of claim 29, wherein the individual suffers from an autoimmune disease.

56. The method of claim 29, wherein the individual is in need of an organ transplant.
